(19) United States
(12) Patent Application Publication
(10) Pub. No.: US 2023/0010107 A1
(43) Pub. Date: Jan. 12, 2023

(54) PROCESS FOR PRODUCING PLATELET-RICH FIBRIN MATRIX FROM PLATELET-RICH PLASMA

(71) Applicant: Lance Liberti, Tampa, FL (US)

(72) Inventor: Lance Liberti, Tampa, FL (US)

(21) Appl. No.: 17/861,764

(22) Filed: Jul. 11, 2022

Related U.S. Application Data
(60) Provisional application No. 63/220,492, filed on Jul. 10, 2021.

(51) Int. Cl. A61K 35/16

(52) U.S. Cl. CPC A61K 35/16

(57) ABSTRACT

A device for producing platelet-rich fibrin matrix (PRFM) from platelet-rich plasma (PRP) in a chemical-free manner. The device includes heating receptacles to receive one or more containers, such as syringes containing the PRP. PRP is to be added to the containers for converting it to PRFM. Uniform heat can be applied to the containers by the heating receptacles for triggering the thrombotic cascade in the PRP contained in the container. After heating for a pre-determined duration, immediately cooling the container by inserting the container in a cooling receptacle also provided in the device.

### CROSS-REFERENCE TO RELATED APPLICATIONS

This application claims priority from a U.S. Provisional Patent Appl. No. 63/220,492 filed on Jul. 10, 2021, which is incorporated herein by reference in its entirety.

### FIELD OF INVENTION

The present invention relates to a device and method for producing platelet-rich fibrin matrix, and more particularly, the present invention relates to a device and the method for producing platelet-rich fibrin matrix from platelet-rich plasma.

### BACKGROUND

Platelets are a naturally occurring component in blood, which plays an important role in the body's healing processes. These platelets contain crucial growth factors that have been shown to stimulate tissue generation and repair. Through a process called centrifugation, these platelets can be isolated from a whole blood sample for therapeutic use. The golden-colored portion obtained after centrifugation is called Platelet Rich Plasma (PRP).

Platelet-Rich Fibrin Matrix (PRFM) is formed from PRP when the thrombotic cascade is triggered within a PRP sample. PRFM is a more dense and highly concentrated product. PRFM is more stable than its more basic PRP counterpart, which allows for a longer active effect within the body; promoting healing for one to two weeks following treatment. This sustained production of new stem cells, renewed blood flow, and increased collagen production delivers unmatched results across many applications within several medical specialties. PRFM treatment is a safe and effective, minimally invasive treatment option utilized today in Dentistry, Orthopedics, Neurology, Internal Medicine, Cosmetic/Aesthetic Medicine, Urology, and Would Care. The PRFM increases endothelial cell generation in wounds and improved the development of new blood vessels in ischemic injuries.

Historically, the process of triggering the thrombotic cascade in PRP for PRFM production was achieved through chemical activation, in which bovine thrombin or calcium chloride is added to a PRP sample. The addition of these compounds heightens patient risk factors, such as allergic reaction, and may have an impact on native blood supply, upon re-injection, leading to increased risk of blood clots and other adverse vascular interactions. The known chemically activated PRFM products have several risk factors which limit the use of PRFM by patients and physicians.

Thus, a need is there for a device and method for producing PRFM from PRP without chemicals, and without the drawbacks of the known method for producing PRFM from PRP.

### SUMMARY OF THE INVENTION

The following presents a simplified summary of one or more embodiments of the present invention to provide a basic understanding of such embodiments. This summary is not an extensive overview of all contemplated embodiments and is intended to neither identify critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a device for chemical free producing platelet-rich fibrin matrix (PRFM) from platelet-rich plasma (PRP).

In one aspect, disclosed is a device for producing platelet-rich fibrin matrix (PRFM) from platelet-rich plasma (PRP), the device includes a housing; one or more heating receptacles configured in the housing, each of the one or more heating receptacles configured to receive one or more containers, each of the one or more heating receptacles integrated with a heating source for applying uniform heat to the one or more containers, the heating source encased within the housing; one or more cooling receptacles, each of the one or more cooling receptacles configured to receive the one or more containers; and a cooling source encased within the housing, the cooling source configured to cool an inner volume of each the one or more cooling receptacles. The heating source is a ceramic heating core layered with a polymer coating. The cooling source is configured to provide cooled air within the inner volume of each one or more cooling receptacles. The device further comprises one or more control buttons and one or more displays, the one or more control buttons are configured to set temperatures and duration for heating and cooling.

A method for producing platelet-rich fibrin matrix (PRFM) from platelet-rich plasma (PRP), the method comprises providing the device; receiving a container of the one or more containers into a heating receptacle of the one or more heating receptacles, the container contains a predetermined amount of PRP; heating the container for a predetermined duration at a pre-determined temperature; upon heating, removing the container from the heating receptacle; upon removing, inserting the container into a cooling receptacle of the one or more cooling receptacles; and cooling the container up to a predetermined temperature.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a front perspective view of a device for producing PRFM from PRP, according to an exemplary embodiment of the present invention.

FIG. 2 is a rear perspective view of the device, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as apparatus and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details. In other instances, well-known structures and apparatus are shown in block diagram form in order to facilitate describing the subject innovation. Moreover, the drawings may not be to scale.

The disclosure is directed to a device and process for producing platelet-rich fibrin matrix (PRFM) from platelet-rich plasma (PRP) in a consistent and autologous fashion, and in a safe and cost-effected manner. Disclosed is a process for producing PRFM from PRP without the use of chemicals and allergens, eliminating patient risk factors, and increasing access to further application and study with PRFM gel.

In one exemplary embodiment, controlled heat and cooling can be used to trigger the thrombotic cascade for converting PRP to PRFM. The disclosed device can provide controlled heat without any harsh conditions. The disclosed device can include a heating source to provide controlled and uniform heat. In one implementation, the heating source can be a ceramic heating core layered with a polymer coating for applying uniform and controlled heat. In a preliminary investigation, the ceramic heating core layered with a polymer coating was found to evenly distribute heat (up to 90 degrees centigrade) to PRP samples, resulting in increased cellular metabolism. This effect naturally triggered the thrombotic cascade. Varying rates of time and heat exposure were found to produce PRFM samples of multiple molecular weights.

It was also discovered that cooling the newly formed PRFM samples could stabilize the gel's consistency. Cooling the PRFM samples allow for extended handing time by the physician without the inconsistencies associated with chemical activators in which the thrombotic cascade continues unchecked. The extended handling time can further reduce the risk of sample misuse in a clinical setting.

In one exemplary embodiment, the ceramic heating core layered with a polymer coating and airflow cooling can naturally trigger the thrombotic cascade in PRP samples; increasing the cellular metabolism of the samples and producing a highly concentrated PRFM gel. The reliable heating and cooling chambers can disperse the desired heating evenly across samples. The resulting PRFM is a smooth gel consistency that allows for PRFM injection through needles and cannulas of various sizes.

In one exemplary embodiment, optimizations were made both for the heating element and coatings to reduce the risk of injury through improper handling of the heating elements. Additionally, the airflow cooling elements can be optimized to reduce noise output, increase air circulation within the cooling chamber, and lower energy consumption. A user interface for visualization of device settings and controls can also be provided. The interface can be presented on a large screen, such as an LCD.

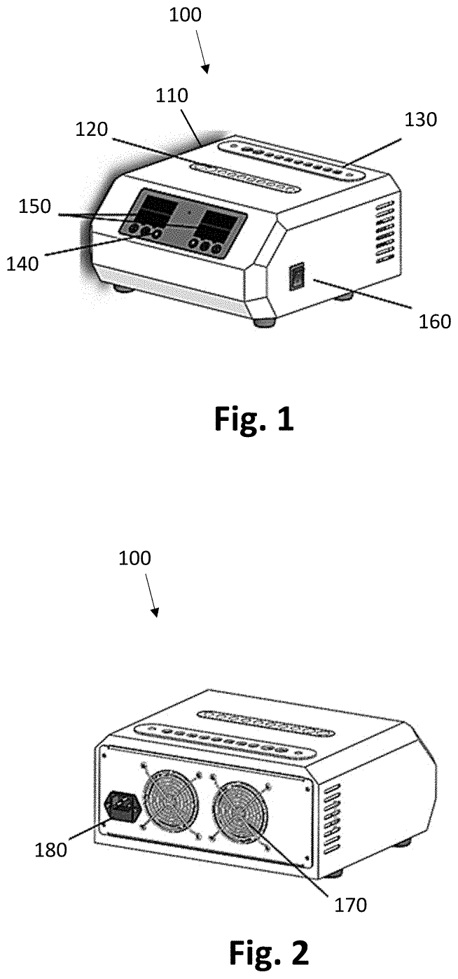

Referring to FIG. 1 which shows an exemplary embodiment of the device 100 for PRFM production from PRP. The device 100 includes a housing 110 that encases a heating source (not shown). The heating source can be a ceramic heating core layered with a polymer coating. The device can further include heating receptacles 120 that can receive the containers for heating. One or more of such heating receptacles can be provided as shown in FIG. 1. The container can hold the PRP sample for heating. In one implementation, the container can be a syringe. The depth of the heating receptacles can be such that the PRP sample within the container can be fully encased within the heating receptacle for uniform heating of the PRP.

Also, the device can include one or more cooling receptacles 130 that can receive the container for cooling the PRFM produced from the PRP. The dimensions of the cooling receptacles can be similar to the heating receptacles. The cooling receptacles can also apply uniform cooling to the PRFM in the containers. In one implementation, cooled air can be flushed around the containers. Suitable control buttons 140 can be provided to set different parameters, such as heating temperature and duration. Similarly, controls for setting cooling temperature and rate can also be provided. Suitable one or more displays 150 can also be provided that display the current status and settings. A power switch 160 is also shown that can be used to turn the device on and off. FIG. 2 is a rear perspective view of the device which shows a fan 170 to circulate air for dissipating the heat and a power socket 180.

Suitable sensors can also be provided within the heating receptacles and the cooling receptacles to monitor the process of heating and cooling. The sensors can detect any change in the viscosity, color, and the like parameters for monitoring the progress of the cycle.

Also, disclosed is a novel process to produce PRFM gel from a PRP sample in a consistent, chemical-free, and drug-free manner. The process involves the use of polymerized ceramic heating and airflow cooling technology to trigger the thrombotic cascade in PRP samples; increasing the cellular metabolism of the samples to produce a highly concentrated PRFM gel. The reliable heating and cooling chambers distribute the desired temperature evenly across samples. The produced PRFM samples are a smooth gel consistency that allows for PRFM injection through needles and cannulas of various sizes.

In one exemplary embodiment, 1 ml, 3 ml, or 5 ml syringes of prepared PRP can be inserted into a heating receptacle, and desired temperature can be set on the interface. Once the samples can be heated to the desired temperature at a desired rate and time, the newly formed PRFM samples can be removed from the heating receptacles and inserted into the cooling receptacles for cooling. Cooling the PRFM samples can slow the progression of the thrombotic cascade to produce more stable samples with a longer handling time.

The disclosed process can produce a smooth PRFM gel that allows for injection through a needle or cannula; greatly increasing the number of potentials and realized clinical applications of PRFM gel without any additives. Cooling the PRFM samples slows the thrombotic cascade, allowing for longer physician handling time and reducing product misuse.

What is claimed is:

1. A device for producing platelet-rich fibrin matrix (PRFM) from platelet-rich plasma (PRP), the device comprises:
a housing;
one or more heating receptacles configured in the housing, each of the one or more heating receptacles configured to receive one or more containers, each of the one or more heating receptacles integrated with a heating source for applying uniform heat to the one or more containers, the heating source encased within the housing;
one or more cooling receptacles, each of the one or more cooling receptacles configured to receive the one or more containers; and
a cooling source encased within the housing, the cooling source configured to cool an inner volume of each the one or more cooling receptacles.

2. The device according to claim 1, wherein the heating source is a ceramic heating core layered with a polymer coating.

3. The device according to claim 1, wherein the cooling source is configured to provide cooled air within the inner volume of each of the one or more cooling receptacles.

4. The device according to claim 1, wherein the device further comprises one or more control buttons and one or more displays, the one or more control buttons are configured to set temperatures and duration for heating and cooling.

5. A method for producing platelet-rich fibrin matrix (PRFM) from platelet-rich plasma (PRP), the method comprises:
providing a device comprising:
a housing,
one or more heating receptacles configured in the housing, each of the one or more heating receptacles configured to receive one or more containers, each of the one or more heating receptacles integrated with a heating source for applying uniform heat to the one or more containers, the heating source encased within the housing,
one or more cooling receptacles, each of the one or more cooling receptacles configured to receive the one or more containers, and
a cooling source encased within the housing, the cooling source configured to cool an inner volume of each the one or more cooling receptacles;
receiving a container of the one or more containers into a heating receptacle of the one or more heating receptacles, the container contains a predetermined amount of PRP;
heating the container for a predetermined duration at a pre-determined temperature;
upon heating, removing the container from the heating receptacle;
upon removing, inserting the container into a cooling receptacle of the one or more cooling receptacles; and
cooling the container up to a predetermined temperature.

6. The method according to claim 5, wherein the heating source is a ceramic heating core layered with a polymer coating.

7. The method according to claim 6, wherein the cooling source is configured to provide cooled air within the inner volume of each of the one or more cooling receptacles.

8. The method according to claim 5, wherein the device further comprises one or more control buttons and one or more displays, the one or more control buttons are configured to set temperatures and duration for heating and cooling.
